(19) United States
(12) Patent Application Publication
(10) Pub. No.: US 2018/0128798 A1
(43) Pub. Date: May 10, 2018

(54) METHODS AND SYSTEMS FOR ANALYSING A FLUID MIXTURE

(71) Applicant: CAMBRIDGE SENSOR INNOVATION LIMITED (FORMERLY CAMBRIDGE SENSOR TECHNOLOGIES LIMITED), Cambridge (GB)

(72) Inventor: Mark Edward WILLIAMSON, Cambridge (GB)

(21) Appl. No.: 15/573,593

(22) PCT Filed: May 13, 2016
(86) PCT No.: PCT/GB2016/051398

§ 371 Date: Nov. 13, 2017

(30) Foreign Application Priority Data
May 13, 2015 (GB) 1508175.5

(51) Int. Cl. G01N 33/00; G01N 33/22; G01N 25/18; G01N 27/407; G01N 1/22; G01K 17/00; A62C 4/02

(52) U.S. Cl. CPC G01N 33/0024; G01N 33/225; G01N 25/18; A62C 4/02; G01N 1/2205; G01N 1/2258; G01K 17/00; G01N 27/407

(57) ABSTRACT

A method of obtaining the concentration of one component in a fluid mixture of a plurality of components, comprising leading to one side of a sensing element from a source of said mixture a sample of said mixture, leading to another side of said sensing element from said source another sample of said mixture after having substantially removed said one component therefrom, thereby to cause a monitoring device to which said sensing element is connected to signal the ratio between the concentrations of the one component in the samples at the respective sides of the sensing element.

This invention relates to a method of, and a system for use in, sensing one component in a fluid mixture of a plurality of components, as well as a flame arresting arrangement and an electrochemical oxygen sensor usable in the method and the system.

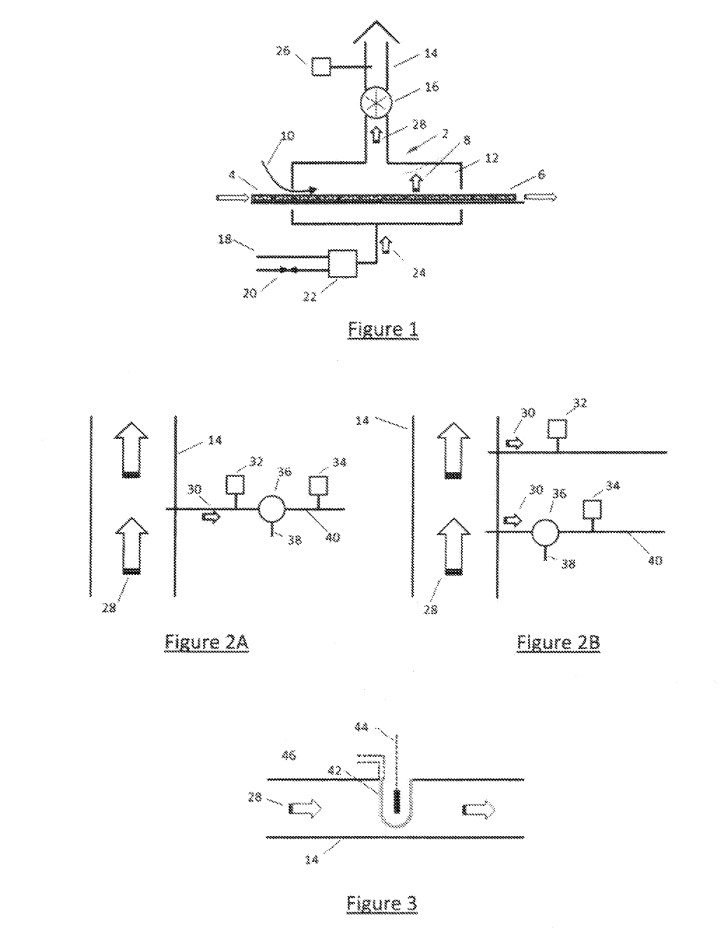

In one known scenario, measurement of the water vapour content of a gas is potentially useful to many process industries. An example is the atmosphere of an oven, kiln, drier, or furnace, as illustrated in FIG. 1 of the accompanying drawings. Referring to FIG. 1, the following reference numerals indicate the following items:—
2 oven, kiln, drier or furnace;
4 incoming material to be processed;
6 outgoing material;
8 moisture and other volatile species leaving the material being processed;
10 excess air drawn into the processing chamber 12 of the oven, kiln, drier or furnace 2;
12 processing chamber;
14 exhaust stack;
16 exhaust fan;
18 air supply to burner 22;
20 fuel supply to burner 22, with shut-off valve;
22 burner;
24 combustion products;
26 moisture vapour sensor;
28 exhaust gases to be measured.

The partial pressure of water vapour in the enclosed atmosphere 12 can often have a significant impact on the final quality of the material being processed, particularly if moisture is to be removed from the material.

In the early stages of processing, before the surface temperature of the material being processed rises above 100° C., the initial rate of moisture loss from the material can be modified by condensation on the surface of the material and/or by manipulation of the mass transfer driving force between the atmospheric boundary layer around the material and the material surface.

In later stages of processing, when the material surface temperature rises above 100° C., atmospheric moisture content within the enclosure 12 no longer directly influences mass transfer (evaporation of water) at the material surface, but does however continue to influence both convection and radiation heat transfer to the material. Infra-red thermal radiation from heat sources within the enclosure 12 (such as the hot enclosure walls, or flames from the burner 22) that is directed at the material being processed is partially absorbed by intervening moisture vapour and reradiated with a uniform hemispherical distribution. For a given enclosure geometry, this effect acts to reduce the net flux of radiation reaching the material. For forced convection heat transfer, arising for example from an array of air jets in the enclosure 12, the partial pressure of water vapour affects the transport properties of the heat transfer medium (viscosity, thermal conductivity, specific heat capacity), and hence the heat transferred.

A common location for a sensor 26 is in the exhaust stack 14, since this contains an average of all conditions that may exist in different locations with the enclosure 12. Any sensor that is to achieve a commercially viable lifetime in an oven, kiln, drier or furnace as mentioned earlier must therefore be sufficiently robust to withstand any conditions that may occur within and around an exhaust stack; in particular:—
Unplanned process disturbances, such as temperature run-aways and fires in the processing enclosure, can result in sudden extreme conditions in the exhaust stack. For example, in a tunnel oven processing food, a fire in one zone of the oven can result in flames being drawn into the stack by the exhaust blower, generating stack temperatures of up to ^{˜}1000° C. for several minutes until the fire is extinguished.
Many processes create dust and/or condensable materials which are extracted through the stack. Sensors must be unaffected by such conditions, or be configured so that they are inherently self-cleaning.
Ambient conditions around the stack may include high temperature (up to 60° C.) and presence of atmospheric dust/oils.

Further consideration are:
The exhaust gases being measured may constitute an explosive mixture, either on a continuous basis or temporarily in the event of a malfunction somewhere in the process. An example is the control system for a gas burner used to heat an oven. When the burner is shut down, the gas supply valve should be closed by the burner interlock system. If this valve does not seal fully, gas can leak into the oven over the duration of the shutdown period, leading to an explosive mixture of air and gas in the oven. It is therefore essential that any sensor does not act as a source of ignition. Known sensor designs address this issue in a number of ways, including:
   Incorporating the sensor power supply into the burner interlock system, so that the sensor has no electrical power when the gas supply valve should be closed. This adds an additional cost to sensor implementation, adds complexity to the process control system, and means that a sensor cannot be tested or recalibrated in situ it must be removed to a remote location and provided with a temporary power supply.
   Positioning a flame arrestor between the process and the sensor. Flame arrestors must be tested and approved to British Standard BS EN12874 (2010), which specifies a maximum testing/operating temperature of 150° C. and 160 kPa absolute. Therefore, in almost all industrial applications, flame arrestors cannot be located in the stack itself, and instead a sample of the gas must be preconditioned (cooled and perhaps reduced in pressure) before it passes through the flame arrestor.
Most ovens, driers and kilns are direct-fired, meaning that the products of combustion from burning of a fossil fuel (e.g. natural gas, methane, propane) enter the processing chamber. In practice, these products of combustion will be diluted by one or more of the following:
   i. Excess air fed to the burner system(s) (gas burners are typically set to run with ^{˜}10% excess air to ensure trouble-free operation and minimal soot/CO formation across the full turndown range of the burner);
   ii. Excess air drawn into the enclosure from the surrounding room by the exhaust fan;
   iii. Moisture evaporated from the material being processed;
   iv. Volatile organic compounds (VOC) evaporated from the material being processed;
   v. Gaseous species from reactions that have occurred in the material being processed, e.g. NH₃;
   vi. Unburnt fuel from a badly adjusted burner system; and
   vii. CO from incomplete combustion.

The species arising from iv, v, vi, and vii together can be classified as oxidisable species. In order to be effective in the widest possible range of industrial applications, a sensor therefore ought not to:
rely on the N₂:O₂ ratio of ^{˜}79/21 that is found in the atmosphere; and
be affected by the presence of oxidisable species.
Some process industries have limited technical resources effectively to support and maintain complex sensors. Any sensor that requires frequent recalibration (>1/year), or that relies on small pumps/fans to withdraw a sample continuously from the stack, or that has a filter requiring monitoring/cleaning, is less likely to gain acceptance by end users in these industries.

In a process application where the O₂/N₂ ratio in the processing chamber is that of air, and where there are no other significant atmospheric components other than water vapour, a single oxygen sensor has been used to measure the volume fraction of water vapour indirectly, by sensing the volume fraction of O₂, and using the relationship:

$${v}_{{H}_{2} O} = 1 - \frac{{v}_{{O}_{2}}}{0.21} \qquad ( Equation 1 )$$

v_{H2O}=volume fraction of water vapour
v_{O2}=volume fraction of oxygen

In direct-fired processes, two oxygen sensors have been employed as shown in FIG. 2A or 2B, using the relationship:

$${v}_{{H}_{2} O} = 1 - \frac{{v}_{{O}_{2} ( wet )}}{{v}_{{O}_{2} ( dry )}} ( 1 - \frac{{p}_{sat ( {T}_{con} )}}{{P}_{s}} ) \qquad ( Equation 2 )$$

v_{H2O}=volume fraction of water vapour
v_{O2(wet)}=volume fraction of oxygen in wet gas
v_{O2(dry)}=volume fraction of oxygen in dry gas
T_{con}=temperature (Kelvin) of condenser used as a drier
pₛₐₜ=saturation vapour pressure of water vapour at T_{con} (Pa)
Pₛ=Total absolute pressure of sample gas (Pa)

Referring to FIGS. 2A and 2B, the following reference numerals indicate the following items:—
14 stack or duct;
28 gases to be measured;
30 sample gas stream;
32 WET oxygen measurement;
34 DRY oxygen measurement;
36 moisture remover;
38 water disposal;
40 WET sample stream.
pₛₐₜ is a known function of T_{con}
T_{con} must be measured
Pₛ must be measured, and is usually assumed to be same for both oxygen sensors and equal to the process pressure, since the pressure drop in the sample line between them can be made negligible.

Zirconia oxygen sensors are a well-proven sensor technology used extensively in the combustion management systems of motor vehicles where they have been proven to withstand the harsh conditions associated with mounting in exhaust manifolds. They have also been used for the indirect measurement of partial pressure of water vapour, as for the sensors 32 and 34 in FIGS. 2A and 2B.

In these sensors, a ZrO₂/Yt₂O₃ ceramic element (often in the form of a cylindrical closed-end thimble) is coated on each side with a porous platinum catalytic layer and heated to a temperature of ^{˜}700° C. FIG. 3 shows an example, wherein the following reference numerals refer to the following items:—
28 sample gas to be measured;
14 stack or duct;
42 ZrO₂/Yt₂O₃ ceramic thimble with porous platinum catalytic coating on inside and outside;
44 heater;
46 ambient atmosphere (reference gas).

Oxygen in the sample gas 28 and the reference gas 44 dissociates at the respective catalytic layers, as shown in Equation (3). If there is a difference in O₂ concentrations between the reference gas 44 and the sample gas 28 then we observe a) diffusion of O⁻⁻ ions through the porous ceramic and b) an EMF (voltage) across the ceramic.

O₂⊇20⁻⁻+4e⁻ (Equation 3)

The magnitude of this EMF is described by the Nernst equation:

$$E = \frac{{RT}_{s}}{zF} ln ( \frac{{p}_{{O}_{2} ( sample gas )}}{{p}_{{O}_{2} ( reference gas )}} ) + {V}_{0} \qquad ( Equation 4 )$$

And applying Dalton's law:

$$E = \frac{{RT}_{s}}{zF} ln ( \frac{{P}_{s} \cdot {v}_{O 2 ( sample gas )}}{{P}_{atm} \cdot {v}_{O 2 ( reference gas )}} ) + {V}_{0} \qquad ( Equation 5 )$$

p_{(O2reference gas)}=partial pressure of O₂ in reference gas (Pa)
p_{(O2sample gas)}=partial pressure of O₂ in sample gas (Pa)
Pₛ=absolute pressure of sample gas (Pa)
Tₛ=temperature (Kelvin) of porous ceramic inside the sensor
R=universal gas constant (8.314 J/mol Kelvin)
z=4 (ref. Equation 3)
F=Faraday constant (96487 C/mol)
V₀=zero offset voltage (V)
E=sensor output voltage (V)

The zero offset voltage (V₀) is due mainly to any differences in temperature that may exist between the two sides of the ceramic. For this reason, many existing zirconia sensors generate a small but significant negative EMF when there is no difference in oxygen partial pressure across the sensor.

The reference gas is typically chosen as ambient air, for convenience.

Any oxidisable species approaching either of the catalytic surfaces will be oxidised immediately, resulting in a lower partial pressure gradient of oxygen across the ceramic as described in Equation (6).

$$E = \frac{{RT}_{s}}{zF} ln ( \frac{{P}_{s} \cdot {K}_{sample gas} \cdot {v}_{O 2 ( sample gas )}}{{P}_{atm} \cdot {K}_{reference gas} \cdot {v}_{O 2 ( reference gas )}} ) + {V}_{0} \\ {K}_{sample gas} = correction factor for sample gas oxidisable species content ( 0 < K < 1 ) \\ {K}_{reference gas} = correction factor for reference gas oxidisable species content ( 0 < K < 1 ) \qquad ( Equation 6 )$$

K_{sample gas}=correction factor for sample gas oxidisable species content (0<K<1)

K_{reference gas}=correction factor for reference gas oxidisable species content (0<K<1)

This effect is usually negligible on the reference (air) side but often significant on the sample gas side.

Now for a system with two zirconia sensors, as shown in FIG. 2A or 2B, combining Equation (6) with Equation (2) we find:

$$\frac{zF}{R} ( \frac{{E}_{wet} - {V}_{0 ( wet )}}{{T}_{s ( wet )}} - \frac{{E}_{dry} - {V}_{0 ( dry )}}{{T}_{s ( dry )}} ) = ln ( \frac{{K}_{wet} {v}_{O 2 ( wet )}}{{K}_{dry} {v}_{O 2 ( dry )}} ) \qquad ( Equation 7 )$$

and combining equation (7) with equation (2):

$${v}_{H 2 O} = 1 - \frac{{K}_{dry}}{{K}_{wet}} ( 1 - \frac{{p}_{sat ( {T}_{con} )}}{{P}_{s}} ) exp [ \frac{zF}{R} ( [ \frac{{E}_{wet} - {V}_{0 ( wet )}}{{T}_{s ( wet )}} ] - [ \frac{{E}_{dry} - {V}_{0 ( dry )}}{{T}_{s ( dry )}} ] ) ] \qquad ( Equation 8 )$$

The implications of this relationship are that, in order to obtain a correct measurement of the moisture content:
Even if K_{dry} can be assumed to be 1.0, K_{wet} must still be measured. In practise this measurement is impractical since there are a great number of species that could be present in quantities that probably vary with time, and so their combined potential to reduce the oxygen concentration could not be assumed constant or continuously determined. Hence most manufacturers of existing sensors that are based on zirconia cells do not recommend their systems for applications where significant quantities of oxidisable species are present.
The sensor ceramic temperatures, T_{wet} and T_{dry}, must be measured.
The condenser temperature T_{con} and sample gas pressure Pₛ must be measured.
The offset voltages V_{0(wet)} and V_{0(dry)} must be determined, after a stable response condition is reached. Even if after a 200 hour burn-in period V_{0(wet)} and V_{0(dry)} are measured as part of an initial calibration procedure (it is well known that the response of zirconia sensors changes significantly during the first 200 hours of use), these values will change as the sensor ages. The primary causes of sensor ageing are a) deterioration in the output of the heater and b) changes in the emissivities of the metal surfaces surrounding the ceramic.

According to a first aspect of the present invention, there is provided a method of obtaining the concentration of one component in a fluid mixture of a plurality of components, comprising leading to one side of a sensing element from a source of said mixture a sample of said mixture, leading to another side of said sensing element from said source a sample of said mixture after having substantially removed said one component therefrom, thereby to cause a monitoring device to which said sensing element is connected to signal a difference between the samples at the respective sides of the sensing element.

According to a second aspect of the present invention, there is provided a system for use in obtaining the concentration of one component in a fluid mixture of a plurality of components, comprising a forwarding arrangement serving to forward from a source one sample of said mixture, a sensing element arranged to receive the forwarded one sample at one side thereof, as well as to forward from said source another sample of said mixture to another side of said sensing element, a removing arrangement in the path of said other sample towards said sensing element and serving to remove substantially said one component thereof, and a monitoring device connected to said sensing element and arranged to signal a difference between the samples at the respective sides of the sensing element.

Owing to those aspects of the invention, a number of variables in the sensing can each be offset against itself, so simplifying the method and system and providing, over an extended period of time (particularly years) a more enduringly accurate determination of the one component content of the mixture.

According to a third aspect of the present invention, there is provided a flame arresting arrangement comprising a thermally conductive base, sintered particles thermally conductively connected with said base and providing sufficient heat transfer area for quenching of a flame, a heater thermally connected with said base and said particles, a temperature sensor thermally connected with said base and said particles, and a control device which is connected to said temperature sensor and which serves in use to prevent the temperature of said base and said particles from falling below a lower threshold and from rising above a higher threshold.

Owing to that aspect of the present invention, any deleterious effect of the temperature of the sintered particles falling below the lower threshold can be avoided, as can any deleterious effect of that temperature exceeding the upper threshold.

According to a fourth aspect of the present invention, there is provided an electrochemical oxygen sensor comprising a sensing plate and first and second chambers at respective opposite sides of said plate for having conducted therethrough respective differing gaseous samples containing respective differing concentrations of oxygen gas, and a device for detecting an electromotive force across said sides.

The further improved sensing system of the invention has the feature that a single sensor is used, making the system less complex and less expensive to manufacture than existing systems using two sensors.

Owing to that aspect of the present invention, a more accurate determination of the oxygen concentration ratio can be achieved.

This invention is concerned with the sensing of the concentration of one component of a multi-component fluid mixture.

In one particular application, the concentration of one component, which may be difficult or impossible to measure directly, may be determined indirectly using a single measurement of the ratio of the concentration of one of the other components in two streams.

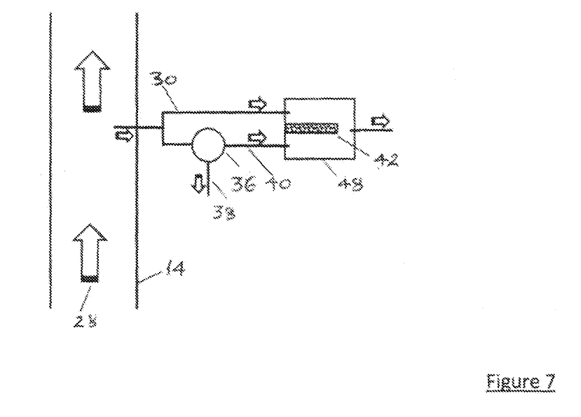

In FIG. 7 a sample of a multicomponent stream (for example containing species a, b, c, d, e, and f) is split into two parallel streams 30 and 40. One stream 40 (referred to as the ‘partial’ stream) passes through a separation process 36, for example a condenser or a molecular sieve, in which only one of the species is removed (for example ‘a’). The other stream 30 (referred to as the ‘complete’ stream) is not modified.

The two streams 30 and 40 then come into contact with a sensing system capable of measuring the ratio of the concentrations of one or more of the species that were not removed in the separation process 36 (for example if species ‘a’ has been removed, an electrochemical cell 42/48 that follows Nernst's law with regard to the concentrations of b, c, d, e, or f, or of a combination of these species). No absolute measurement of these two concentrations is necessary, only the ratio.

For example if species ‘b’ is chosen for removal in order to measure the concentration of species ‘a’:

$${v}_{a} = 1 - \frac{{v}_{b ( complete )}}{{v}_{b ( partial )}} \qquad Equation ( 10 )$$

vₐ=concentration of species ‘a’ in sample (for example expressed as a volume or molar fraction)

v_{b(complete)}=concentration of species ‘b’ in unmodified stream (expressed as a volume or molar fraction)

v_{b(partial)}=concentration of species ‘b’ in modified stream (expressed as a volume or molar fraction)

The advantages of such a sensing system are:
Any detrimental effects that one or more of the species may have on the sensing element 42, that would cause an error in an absolute concentration measurement, cancel out in a ratio measurement since they affect both sides of the sensor equally.
Any changes in the response characteristic of the sensing element 42 over the lifetime of the sensing system (for example loss of sensitivity due to ageing or fouling) cancel out in a ratio measurement and so do not affect the accuracy of the measurement system.

Referring to FIG. 7, the following reference numerals indicate the following items:
14 stack or duct;
28 gases to be measured;
30 complete sample stream;
40 partial sample stream;
36 removal of one or more species from the stream;
38 disposal (or return) of separated specie(s);
48 sensor body;
42 differential sensing element.

Additional improvements can be that, in sensing water vapour content, in place of the condenser used in existing designs, an alternative method (for example a suitable molecular sieve) is used to remove all of the water but none of the oxidisable species. This eliminates the need to know the concentration of oxidisable species on each side of the sensor, since (whatever it is) it is the same for both.

A further improvement can be that the internal construction of the sensor is configured so that there is a negligible temperature difference across the ZrO₂/Yt₂O₃ ceramic both initially and over timescales of a year or more. This eliminates error that would otherwise be caused by the offset voltage changing as the sensor ages.

In order that the invention may be clearly understood and readily carried into effect, reference will be made, by way of example, to the accompanying drawings, in which:—

FIG. 1 shows diagrammatically, an example of a known system for sensing of the water vapour content of exhaust gas from an oven, kiln or drier;

FIG. 2A shows diagrammatically a first version of a known, improved system;

FIG. 2B shows diagrammatically a second version of the known, improved system;

FIG. 3 shows diagrammatically a known zirconia oxygen sensor;

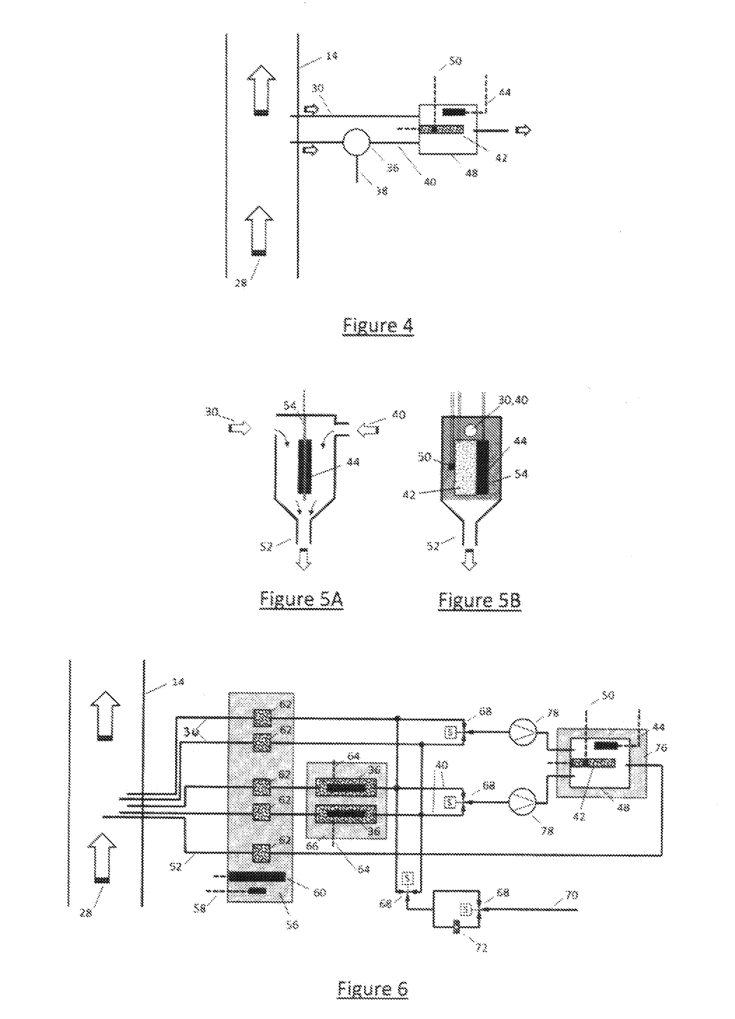

FIG. 4 shows an example of the present invention and constituting a further improvement over the known versions shown in FIGS. 2A and 2B;

FIGS. 5A and 5B show diagrammatically axial sections taken orthogonally relative to each other through a zirconia oxygen sensor constituting an inventive improvement upon the known sensor of FIG. 3;

FIG. 6 shows diagrammatically and in greater detail an example according to the present invention and constituting a development of the inventive example shown in FIG. 4; and

FIG. 7 shows diagrammatically a principle of the present invention.

Referring to FIG. 4, which shows the present invention schematically, the following reference numerals indicate the following items:—
14 stack or duct;
28 gases to be measured;
30 WET sample stream;
40 DRY sample stream;
36 water removal;
38 water disposal;
48 zirconia sensor outer casing;
50 temperature sensor;
44 heater;
42 ZrO₂/Yt₂O₃ ceramic rectangular plate with platinum catalytic coating on inside and outside.

The moisture vapour content of the sample gases from this sensor 50 is then:

$${v}_{H 2 O} = 1 - exp [ \frac{zFE}{{RT}_{s}} ] \qquad ( Equation 9 )$$

This is a very simple relationship between sensor EMF and the sample water vapour content, particularly when compared to Equation (8) which applies to known sensor configurations.

FIGS. 5A and 5B show the zirconia oxygen sensor of FIG. 4 in more detail, with the following reference numbers indicating the following items:—
30 WET sample inlet;
40 DRY sample inlet;
52 sample gaseous returned to process;
50 temperature sensor;
42 ZrO₂/Yt₂O₃ ceramic plate with platinum catalytic coating on inside and outside;
54 dividing plate to create separate DRY and WET measurement chambers, and to support the items 42, 44 and 50;
44 heater element.

The operating temperature of each face of the plate 5 will depend on a heat balance between energy supplied to that face [by (i) the heater 44 and (ii) the incoming sample 52] and energy lost from that face to the surroundings.

Therefore, in this embodiment:
The temperature of the incoming WET and DRY sample gas streams 30 and 40 is arranged to be substantially the same. Differences in flow rate of the WET and DRY samples (due to water removal from the DRY sample) can be shown to have a negligible effect on the heat balance.
The location of the heater element 44 is symmetrical with respect to the two sensing faces of the plate 42, and the whole sensor assembly has a plane of symmetry through the centre of the ceramic plate 42 and the heater 44. This ensures the same radiation convection and conduction heat transfer to each face of the plate 42. In particular, as the emissivities of the various surfaces within the sensor change due to ageing (e.g. tarnishing of the inside of the sensor casing 48) the changes to net radiation heat transfer received by the two faces of the plate 42 will be the same and so will not create a temperature difference between the two faces.
The sensor assembly is insulated to avoid differences in heat loss to the surroundings from the WET and DRY sides of the outer sensor casing 48.

In order to make the sensor suitable for the widest possible range of industrial application, the following issues are addressed:
All sample gas connections to the process to include a flame arrestor;
All sample gas entering the sensor to be filtered. The filters should be self-cleaning, to permit continuous operation of the sensor system for at least a year without operator intervention;
All sample gas species to be returned to the process (in case they are toxic or flammable);
The gas drying system (e.g. a bed of molecular sieve) to be self-regenerating, to permit continuous operation of the sensor system for at least a year without operator intervention;
The travel time for the sample gas from the process to both sides of the sensor to be the same.

The sensor configuration shown in FIG. 6 addresses these requirements. The following reference numerals therein indicate the following items:
14 stack or duct;
28 process gases to be sampled;
30 supply lines for WET sample;
40 supply lines for DRY sample;
52 sample return line;
56 thermally conductive block, losing heat to ambient air;
58 temperature sensor, used in conjunction with heater element 60 to control block 56 at 120° C.;
60 heater element;
62 flame arrestor/filter element;
64 heater element;
36 drier—bed of molecular sieve (e.g. type 3A) to absorb water selectively;
66 thermal insulation;
68 3-way solenoid valve;
701 instrument air supply (e.g. 7 barg);
72 flow restrictor to provide suitable flow for purging driers 36 during regeneration;
50 temperature sensor;
42 ZrO₂/Yt₂O₃ ceramic plate with catalytic porous platinum coating on both faces;
44 heater;
48 outer housing of zirconia sensor;
76 thermal insulation;
78 positive displacement sampling pumps (piezoelectric or similar).

Important features of this embodiment are:
The functionality of the flame arrestor and filter arrangement 62 is combined. Thermally conductive elements 62 (e.g. porous plugs made from small sintered metal spheres, as used for pneumatic air vent silencers) through which the sample gas must pass, provide sufficient heat transfer area for quenching of a flame. These elements are housed in a thermally conductive block 56 that loses heat to its ambient surroundings. The block 56 is controlled at a temperature in the range 100-150° C. using a heater element 60, which ensures that no water vapour condenses before it reaches the zirconia sensor and that the maximum allowable temperature of 150° C. for operation of the flame arresters 62 is not exceeded. The heater element 60 is sized so that under no circumstances can it generate sufficient power to raise the block above 150° C., and hence the system is fail-safe.
Regeneration of the alternating molecular sieve bed driers 36 is combined with cleaning of the filter/flame arrestors 62. A valve system is used to switch automatically between two filter/drier units 62, 64 at regular intervals. The filter/drier unit 62, 64 that is being regenerated is heated to a suitable temperature (typically 175-260° C.) using a heater 36 whilst being purged with a low flow rate of dry air, typically from an instrument air supply 70, which is restricted by an orifice 72. At the end of the regeneration period a much higher pressure of dry air is used (by bypassing the instrument air around the orifice 72) for a relatively short period of time (perhaps 5 seconds) to remove any particles of process material that have accumulated on or inside the filter/flame arrestor 62, and to cool down the drier 36.
The travel times of the WET and DRY samples from the stack 14 to the zirconia sensor 42 should be similar (to within perhaps a second), even though some or perhaps most of the initial WET sample gas volumetric flow rate is removed in the drier 36. This is required to ensure that the gas streams entering each side of the sensor 42 are synchronized, and therefore do not give an erroneous transient response to a step change in stack gas composition. For example, if the gas being sampled is 90% (vol) water vapour, the volumetric flow of the wet sample being drawn from the stack must be approximately ten times that of the DRY sample to ensure that the travel time between the stack and the zirconia sensor are the same. This can be achieved, for example, by:
   (i) Using two identical positive displacement (i.e. constant volume) sampling pumps 78, positioned as shown in FIG. 6. This ensures that the travel time between the drier 36 and the zirconia sensor 42 for the DRY sample is the same as that for the WET sample from a point adjacent to the drier to the zirconia sensor 42.
   (ii) Arranging for the travel times between the stack 14 and the drier 36 to be as short as possible (of the order of a second). This can be achieved by appropriate sizing of the pumps 78 and the diameters of the sampling tubes (30,40). Any residual lack of synchronicity in analysis of the two samples can be smoothed using suitable time-averaging software algorithms (typically over a period of 2-5 seconds).
The ends of the various sample gas pipes 30, 40 and 52 are arranged in the stack 14 so that sample gas or instrument air passing through one or more of them back into the stack 14 does not cause an unrepresentative sample of the exhaust gas 28 to be drawn into the sensor through the pipes 30 and 40.

In industrial applications where there are negligible quantities of oxidisable species, or where a less accurate measurement of water vapour content will suffice for process control purposes, the system shown in FIG. 6 may be used with a conventional condenser in place of the molecular sieve driers 36 to remove water from the DRY gas sample. It would be necessary, however, to measure the condenser temperature.

If, in addition, there is no need for filtration of the incoming sample gases, then the use of a condensing system in place of molecular sieve driers 36 will eliminate the requirement for instrument air and associated solenoid valves 68.

Finally, if all of the above conditions exist, and in addition the sample gas can never be a combustible mixture, then the flame arrestor/filter assembly can be omitted.

1. A method of obtaining the concentration of one component in a fluid mixture of a plurality of components, comprising leading to one side of a sensing element from a source of said mixture a sample of said mixture, leading to another side of said sensing element from said source another sample of said mixture after having substantially removed said one component therefrom, thereby to cause a monitoring device to which said sensing element is connected to signal the ratio between the concentrations of the one component in the samples at the respective sides of the sensing element.

2. A method according to claim 1 and further comprising maintaining no, or at most a substantially negligible, temperature difference between said one side and said other side.

3. A method according to claim 1, wherein said mixture is a product of combustion, said method further comprising arresting, in a flame arresting arrangement, any flame progressing in one or both of said samples towards said sensing element

4. A method according to claim 3, wherein said arresting is performed by thermal conduction at a temperature of no less than 100° C.

5. A method according to claim 4, wherein the temperature at which said arresting is performed is not higher than 150° C.

6. A method according to claim 1 and further comprising filtering solids from one or both of said samples before the sample(s) reach said sensing element.

7. A method according to claim 6, wherein said arresting is performed by alternate flame arrestors of which one is effective whilst another is being cleaned, and wherein also said filtering is performed by said alternate flame arrestors.

8. A method according to claim 1 and further comprising returning the samples after sensing to said source of said gaseous medium.

9. A method according to claim 1, wherein the travel time of said one sample to said one side is substantially equal to the travel time of said other sample to said other side.

10. A method according to claim 9, wherein said travel times are less than the response time of said sensing element.

11. A method according to claim 1, wherein said mixture is a gaseous medium and said one component is water vapour, wherein the removal of the water vapour is performed by passing said other sample through a moisture-removing arrangement.

12. A hod according to claim 11, wherein said other component is oxygen and said sensing element is an oxygen sensing element.

13. A method according to claim 12 and further comprising removing, at said moisture-removing arrangement, substantially all of the moisture in said other sample, but substantially none of any oxidisable species.

14. A method according to claim 11, wherein said moisture-removing arrangement comprises first and second moisture-removing devices of which one is effective whilst another is being re-generated.

15. A method according to claim 14, wherein the switching-over from effectiveness of one of said flame arrestors to cleaning thereof and from one of said moisture-removing devices to regeneration thereof occurs substantially simultaneously.

16. A method according to claim 14, wherein the regeneration of the moisture-removing arrangement is performed by heating the same to evaporate condensed water vapour therein and causing a flow of dry gas to occur therethrough in a direction towards said source.

17. A method according to claim 16, wherein a cooling gas flow is passed through the heated, ineffective, moisture-removing device to cool the same following the re-generation thereof and said flow is passed towards said source through the ineffective flame arrestor to clean the same.

18. A system for use in obtaining the concentration of one component in a fluid mixture of a plurality of components, comprising a forwarding arrangement serving to forward from a source one sample of said mixture, a sensing element arranged to receive the forwarded one sample at one side thereof, as well as to forward from said source another sample of said mixture to another side of said sensing element, a removing arrangement in the path of said other sample towards said sensing element and serving to remove substantially said one component thereof, and a monitoring device connected to said sensing element and arranged to signal the ratio between the concentrations of the one component in the samples at the respective sides of the sensing element.

19. A system according to claim 18, and further comprising a temperature-adjusting device serving to maintain substantially no, or at most a negligible, temperature difference between said one side and said other side.

20. A system according to claim 18, said gaseous medium is a product of combustion and said source is an exhaust channel.

21. A system according to claim 20, and further comprising a flame arresting arrangement arranged in the flow of said samples between said stack and said sensing element.

22. A system according to claim 21, wherein said flame arresting arrangement serves as a filtering arrangement for solids in said one sample and said other sample.

23. A system according to claim 22, wherein said flame arresting arrangement comprises a thermally conductive base, a sintered particles arrangement thermally conductively connected with said base and providing sufficient heat transfer area for quenching of a flame, a heater thermally connected with said base and said particles, a temperature sensor thermally connected with said base and said particles, and a control device which is connected to said temperature sensor and which serves in use to prevent the temperature of said base and said particles from falling below a lower threshold and from rising above a higher threshold.

24. A system according to claim 23, wherein said heater has a heating capability of said flame arresting arrangement no higher than said higher threshold.

25. A system according to claim 24, wherein said higher threshold is 150° C.

26. A system according to 21, wherein said flame arresting arrangement comprises first and second flame arrestors, said system serving to switch the first arrestor to a cleaning condition and the second arrestor to an effective condition, and vice-versa, alternatingly.

27. A system according to claim 18 and further comprising return ducting extending downstream from said sensing element towards said source for leading said samples to said source.

28. A system according to claim 27, wherein said return ducting debouches in said exhaust channel at a location such that the gaseous material exiting therefrom substantially avoids the mixture being sampled therefrom.

29. A system according to claim 18, wherein said sensing element comprises a sensing plate and first and second chambers at respective opposite sides of said plate for having said one sample and said other sample respectively conducted therethrough, said monitoring device serving to detect an electromotive force across said sides.

30. A system according to claim 18, wherein said mixture is a gaseous medium and said one component is water vapour.

31. A system according to claim 30, wherein said sensing element is an oxygen sensing element.

32. A system according to claim 30, wherein said removing arrangement comprises a molecular sieve arrangement of a sieving size to obstruct the passage of water vapour molecules therethrough but to allow the passage therethrough of smaller molecules.

33. A system according to claim 32, wherein said removing arrangement comprises first and second removers, said system serving to switch the first remover to a regeneration condition and the second remover to an effective condition, and vice-versa, alternatingly.

34. A system according to claim 33, and further comprising a heating arrangement serving to heat the relevant ineffective, remover and supply ducting for supplying dry gas to that remover, to re-generate the same.

35. A system according to claim 34, wherein said supply ducting serves to supply cooling air to said relevant, ineffective remover following regeneration thereof and to supply said cooling air to the relevant, ineffective flame arrestor to clean the same.

36. A flame arresting arrangement comprising a thermally conductive base, sintered particles thermally conductively connected with said base and providing sufficient heat transfer area for quenching of a flame, a heater thermally connected with said base and said particles, a temperature sensor thermally connected with said base and said particles, and a control device which is connected to said temperature sensor and which serves in use to prevent the temperature of said base and said particles from falling below a lower threshold and from rising above a higher threshold.

37. A flame arresting arrangement according to claim 36, wherein said lower threshold is 100° C.

38. A flame arresting arrangement according to claim 36, wherein said heater has a heating capability of said flame arresting arrangement no higher than said higher threshold.

39. A flame arresting arrangement according to claim 38, wherein said higher threshold is 150° C.

40. A flame arresting arrangement according to claim 36, and comprising first and second flame arrestors switchable between a cleaning condition for the first arrestor and an effective condition for the second arrestor, on the one hand, and vice-versa, on the other hand, alternatingly.

41. An electrochemical oxygen sensor comprising a sensing plate and first and second chambers at respective opposite sides of said plate for having conducted therethrough respective differing gaseous samples containing respective differing concentrations of oxygen gas, and a device for detecting an electromotive force across said sides, said sensing plate and said chambers being arranged substantially symmetrically with substantial sources and sinks for heat transfer.

42. A sensor according to claim 41 and in the form of a zirconia oxygen-sensing element.
